(19) United States
(12) Patent Application Publication
(10) Pub. No.: US 2018/0197637 A1
(43) Pub. Date: Jul. 12, 2018

(54) CONSUMER WELLBEING ALGORITHM

(71) Applicant: Dermal Diagnostics Limited, Leicestershire (GB)

(72) Inventor: Dewan Fazlul Hoque Chowdhury, Leicestershire (GB)

(21) Appl. No.: 15/740,725

(22) PCT Filed: Apr. 11, 2016
(86) PCT No.: PCT/GB2016/051002

§ 371 Date: Dec. 28, 2017

(30) Foreign Application Priority Data
Jul. 1, 2015 (GB) 1511581.9

(51) Int. Cl. G16H 50/30; A61B 5/00

(52) U.S. Cl. CPC G16H 50/30; G16H 40/63; A61B 5/6801

(57) ABSTRACT

The disclosure relates to a method for monitoring the health or wellbeing of a user, comprising: receiving data associated with an output parameter resulting from a physiological response of the user to an input parameter; and enabling the data to be viewed by the user only after a period of time has elapsed such that the viewable data is not indicative of a real time value of the output parameter.

The disclosure relate to devices and methods in the field of health and general wellbeing monitoring. In particular, although not exclusively, the disclosure relates to a device and associated method for monitoring the health or wellbeing of a user.

Wearable devices allow users to monitor their health and general wellbeing, empowering the user to make decisions that can help improve their general state of health. This also applies to medical devices designed for routine patient monitoring, either continuously or intermittently. Despite the benefits of such devices, users rapidly lose interest or are not motivated to use such devices to enhance their health and wellbeing, often even in cases where the user may have a chronic condition with poor long term prognosis such as diabetes. Other conditions or indications that can aid general wellness and healthy lifestyles, include:
weight management
physical fitness
stress management
mental acuity
sleep management
self esteem

According to a first aspect of the disclosure there is provided a method for monitoring the health or wellbeing of a user, comprising:
receiving data associated with an output parameter resulting from a physiological response of the user to an input parameter; and
enabling the data to be viewed by the user only after a period of time has elapsed such that the viewable data is not indicative of a real time value of the output parameter.

The period may be a pre-defined period. The input parameter may include one of a time, duration, intensity or quantity of: food or liquid consumption; medicine consumption; exercise or physical movement. The output parameter may include one or more of: a blood glucose level; calorific burn rate; calorific intake; heart rate; breathing rate; expiratory and inspiratory volumes; lactic acid levels; drug levels; core temperature; skin temperature; perspiration; and blood pressure.

The input parameter may be a quantity of food or liquid consumed by the user. The pre-defined period may be a minimum of 1 hour starting from the cessation of food or liquid consumption.

The input parameter may be a quantity of food or liquid consumed by the user. The period may be determined in accordance with a sugar level falling back to a normal or rest range, as determined using sensors on or in the user's body.

The input parameter may be a duration or intensity of exercise performed by the user. The pre-defined period may be a minimum of 5 minutes from cessation of exercise.

The input parameter may be a duration or intensity of exercise performed by the user. The period may be determined in accordance with the output parameter falling back to a rest level.

The method may further comprise devising an input parameter plan for a subsequent period. The input parameter plan may be determined algorithmically based on historic performance. The input parameter plan may be determine based on user-desired output parameters. A computer generated value indicating the extent of wellbeing of the user may be determined for the user based on the input and/or output parameters.

The method may comprise encrypting, at a first device, the data regarding the output parameter to provide encrypted data. The method may comprise sending, from the first device to the second device, the encrypted data and unencrypted data regarding non-confidential user information. The method may comprise using, at the second device, the unencrypted data to select whether to decrypt the encrypted data.

According to a further aspect of the disclosure there is provided a device for monitoring the health of a user, the device configured to:
receive data associated with an output parameter resulting from a physiological response of the user to an input parameter; and
enable the data to be viewed by the user only after a period of time has elapsed.

The apparatus may be configured to perform any method step described with reference to the first aspect of the disclosure or have any feature described with reference to the first aspect. For example, the period may be pre-defined. The input parameter may include one of: a time, duration, intensity or quantity of: food or liquid consumption; medicine consumption; exercise or physical movement. The output parameter may include one or more of: a blood glucose level; calorific burn rate; calorific intake; heart rate; breathing rate; expiratory and inspiratory volumes; lactic acid levels; drug levels; core temperature; skin temperature; perspiration; blood pressure.

The device may comprise a sensor for placing on or in a body of the user and for determining the data associated with a physiological response of the user. The device may be a wearable device. The may be further configured to devise an input parameter plan for a subsequent period.

According to a further aspect of the disclosure these is provided a method for sharing, between a first device and a second device, data regarding medical or personal information associated with a first user, in which the data contains confidential and non-confidential user information, the method comprising:
encrypting, at the first device, the data regarding the confidential user information to provide encrypted data;
sending, from the first device to the second device, the encrypted data and unencrypted data regarding the non-confidential user information;
using, at the second device, the unencrypted data to select whether to decrypt the encrypted data.

The encrypted data may be sent substantially simultaneously with the unencrypted data. The confidential information may include one or more of: user input parameters; input parameter plans; and user output parameters of the first user.

The non-confidential information may include specific user characteristics of the first user. The specific user characteristics of the first user may be compared with specific user characteristics of the second user in order to determine whether or not to decrypt the encrypted data. The specific user characteristics may include one or more of age, height, weight, race, gender, disease state, body mass index.

There may be provided a computer program, which when run on a computer, causes the computer to configure any apparatus, including a circuit, computing unit, processor or device disclosed herein or perform any method disclosed herein. The computer program may be a software implementation, and the computer may be considered as any appropriate hardware, including a digital signal processor, a microcontroller, and an implementation in read only memory (ROM), erasable programmable read only memory (EPROM) or electronically erasable programmable read only memory (EEPROM), as non-limiting examples. The software may be an assembly program.

The computer program may be provided on a computer readable medium, which may be a physical computer readable medium such as a disc or a memory device, or may be embodied as a transient signal. Such a transient signal may be a network download, including an internet download.

One or more embodiments of the invention will now be described, by way of example only, and with reference to the accompanying figures in which:

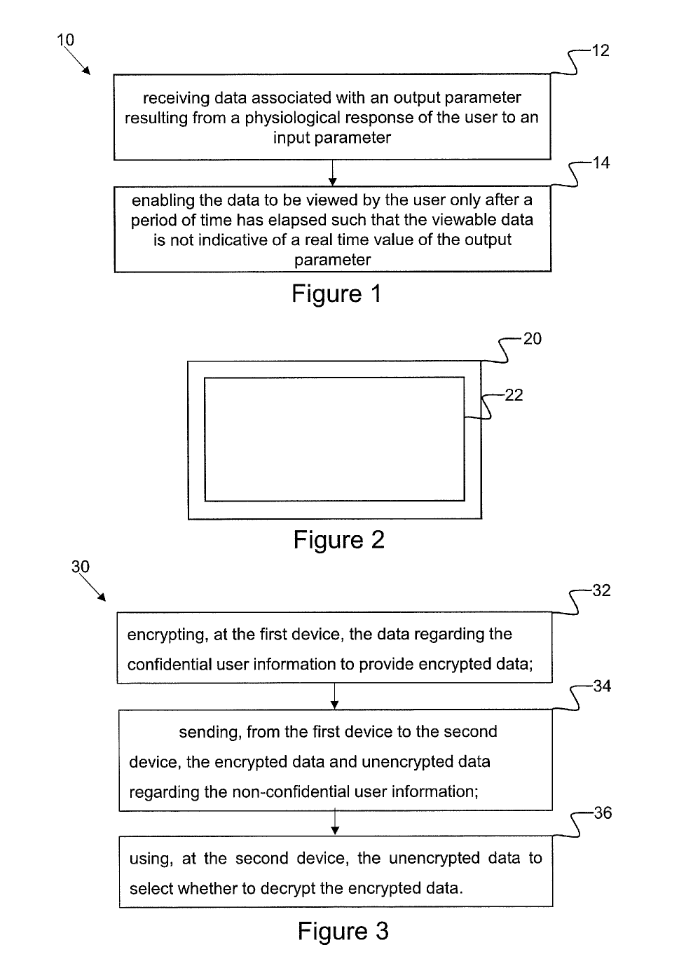

FIG. 1 illustrates a method for monitoring the health or wellbeing of a user;

FIG. 2 illustrates a block diagram for an apparatus for monitoring the health or wellbeing of a user;

FIG. 3 illustrates a method for sharing, between a first device and a second device, data regarding medical or personal information; and

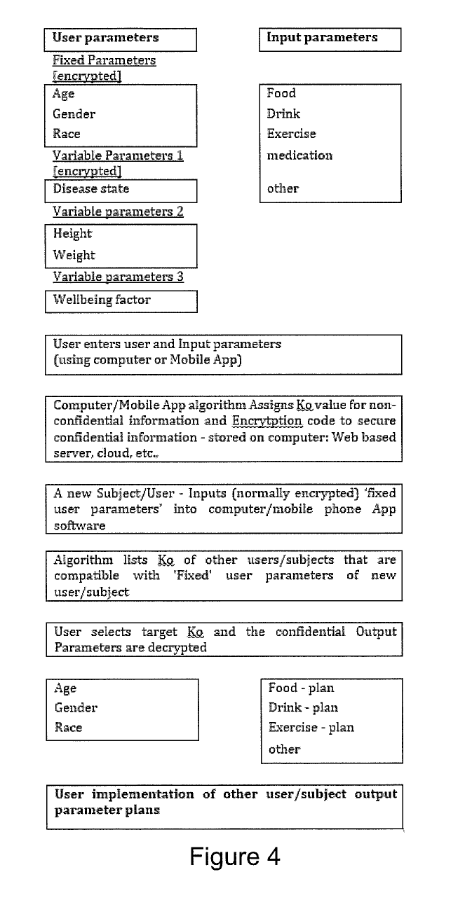

FIG. 4 illustrates an example of a method that relates to the method of FIG. 3.

FIG. 1 illustrates a method 10 for monitoring the health or wellbeing of a user. The method comprises receiving 12 data associated with an output parameter resulting from a physiological response of the user to an input parameter. The data is enabled 14 to be viewed by the user only after a period of time has elapsed such that the viewable data is not indicative of a real time value of the output parameter.

FIG. 2 illustrates a block diagram for a device 20 for monitoring the health of a user. The device 20 comprises a computer 22 that may be configured to perform the method of FIG. 1. The computer may be a general purpose computer, a mobile computer, a web based server, cloud or cloud computer, for example,

This device of FIG. 1 and method of FIG. 2 seek to overcome the issue of patient motivation, and enhance the learning process, or user training process by preventing the user from viewing data in real time, and allowing data only to be viewed after a pre-defined period has elapsed. This pre-defined period is a minimum of the time required after which the given effect or measured parameter can no longer be altered in real time. For example if measuring calorie consumption in the form of blood sugar level, then the device will not allow the user to view the glucose levels and trends until approximately 1 hour post the calorific intake, since it is expected that the glucose levels will be modulated by an individuals physiological system such that any intervention after 1 hour will lead to a new discrete sugar level trend completely independent of the historic real time trend that occurred during the 1 hour prior. This acts to psychologically motivate a user to continuously improve their ability to modulate their day-to-day activities and lifestyle choices, with a view to achieving their set target. This provides an added benefit over and above real time monitoring, in that firstly the user seeks to use their knowledge of historic events to improve future events, and secondly it provides an intellectually rewarding stimulus which real time monitoring cannot provide in light of the interventions a person is able to make without any significant intellectual engagement or predictive thought processes in real time monitoring.

A method of retrospective evaluation of the output-parameters of historic input-parameters, whereby the output-parameters can only be retrieved or viewed or evaluated after such time that any manual or automated adjustment of the input-parameter is no longer able to affect or influence the output-parameter of a previous episode. The previous episode is defined in the case of food intake a period over which a finite amount of food or drink was consumed following a finite rest period, and in the case of exercise the period over which a finite amount of exercise was performed followed by a finite period of rest, whereby in each case the next episode is a distinctly new episode, that does not substantially overlap in terms of physiological response of the subject to that discrete previous episode. A previous episode may also be defined as a minimum of 3 half lives of the physiological response to the input parameters, thus approximately when the response is 12.5% of the initial peak response. The input parameter includes but is not limited to, time, duration/intensity/quantity (where appropriate) of:
food consumption, solids or liquid
consumption of medicine
exercise or physical movement

Output Parameters include but is not limited to:
blood glucose level (in blood or interstitial fluid or sweat)
calorific burn rate
calorific intake
heart rate
breathing rate, and expiratory and inspiratory volumes
lactic acid levels (in blood or interstitial fluid, or sweat)
drug levels (in blood or interstitial fluid, or sweat)
body temperature [core temperature or skin temperature]
perspiration
blood pressure

Following the input parameter, the period that must elapse prior to the device triggering the release and access to the data that has been historically gathered as the output parameters values, defined as the ‘period elapsed’ is determined as follows:

Input Parameter:

Consumption of food/liquids—period elapsed: either
pre-programmed value of minimum 1 hour post cessation of input parameter (designed to provide sufficient buffer to allow output parameter to revert to its normal or rest value, following the input parameter), or
self-regulated determination of period elapsed—whereby the rise in sugar level falls back to its normal or rest range, as determined using appropriate sensors on or in the body.

Exercise—period elapsed: either
pre-programmed value of a minimum of 5 minutes from cessation of input parameter, or
self-regulated determination of period elapsed—whereby an output parameter such as heart rate, breathing rate, perspiration, lactic acid concentration, glucose level, or body temperature falls back to its rest level, as determined using appropriate sensors on or in the body, or by taking biological samples such as finger prick blood sample which is then measured using an external device.

The rest level is defined here as the level/value at which the output parameter was at—during one previous measurement period whereby the measurement period is a continuous measurement period of at least 30 minutes, whereby at least 2 recordings of the output parameter are made during this measurement period, or where the measurement is taken intermittently then it is defined as at least 2 measurements taken over a period of at least 30 minutes.

The rest level may be altered and redefined by the algorithm, whereby the rest period may be set as a value that is determined from intermittent rest measurements taken on multiple days. Alternatively the rest level may be taken as the normal physiological human rest level for a human subject, based on values established in literature.

After accessing the retrospective data the user may devise an input parameter plan for a subsequent period. These may constitute a value that is determined by the user manually, or suggested by the algorithm based on historic performance, or be determine based on user-desired output parameters that the user may enter manually. A computer generated Ko value will be determined for the user based on the input and output parameters, indicating the extent of wellbeing of the user.

FIG. 3 illustrates a method 30 for sharing, between a first device and a second device, data regarding medical or personal information associated with a user, in which the data contains confidential and non-confidential user information. The method 30 comprises encrypting 32, at the first device, the data regarding the confidential user information to provide encrypted data. The encrypted data and unencrypted data regarding the non-confidential user information are sent 34 from the first device to the second device. The unencrypted data are used 36 at the second device to select whether to decrypt the encrypted data.

A method, such as that described with reference to FIG. 3, may allow the simultaneous sharing of encrypted and non-encrypted data, that is medically or otherwise sensitive, and non-sensitive, respectively, allowing users to acquire and decide on the basis of the non-encrypted data which encrypted data to view to learn from input plans that may be relevant to their own wellbeing. This promotes and encourages the public sharing of otherwise sensitive and confidential data that a user would not otherwise willingly share for fear of public disclosure of users identity. The simultaneous encrypted and non-encrypted data prevents such inadvertent public disclosure and instills confidence in a user, broadening the extent to which users share health hand wellbeing data globally, for the benefit of users, healthcare providers and general communities.

The user will be able to receive data in relation to other users, in terms of input parameters, and resulting output parameters, based on their Ko, that is calculated based on their specific user characteristics—including: age, height, weight, race, gender, disease state, and other factors that can affect the value of the output parameters for a given subject. Users may choose to adopt input parameters of other users whose Ko value falls within a pre-defined range relevant to the users Ko value. This predefined range for the Ko value will remain dynamic, as it is constantly refined and adjusted automatically by the algorithm to provide a meaningful target for users of similar Ko values. The Ko value will encrypt the input parameters that have lead to the output parameters for a given user, and the algorithm will automatically determine the output parameters for that user based on an encryption code that is sent to others by the user wanting to share his/her input parameters. This may be achieved by standard computer data encryption methods, and is designed to preserve the identity of the patient, and potential sensitive medical information. The Ko value may be the body mass index (BMI) of the person, or it may be some other parameter determined based on a combination of BMI and general feeling of wellbeing/wellness, for example BMI multiplied by a number between 1 and 10, where 10 is a general feeling of excellent health and wellbeing and 1 being the opposite end of the spectrum.

The output parameters are measured by a wearable device or an intermittent point of use device such as continuous blood glucose monitor/temperature/heart rate/blood pressure, electro cardiogram monitor, or non-continuous diagnostic meter respectively, or other type of device that are well established in the literature, worn on one or more place of the body, or used on an ad hoc basis, recording data as per the output parameters discussed above.

Retrospective retrieval of that data may be directly from the device itself, on a screen or other suitable user interface, or it may be ported physically by wire connection or wirelessly to another device such as a smart phone or tablet device. The input parameters for target Ko values (based on other users) may be received wirelessly or via email, and decrypted on receipt of the encryption code. Sensitive data such as age, gender, race, can therefore be shared securely and discreetly only where a user can identify with another target Ko value that he/she wishes to target. User will enter their own personal details, such as gender, race, age, etc., which may be deemed to lead to different interpretations and calculations of general overall wellbeing, and this will allow the algorithm to select matching encrypted data (based on age, gender, race, etc.,) and Ko values that the user may then access and decrypt the Input parameter plans that he/she may then implement. An example of this process is illustrated in the schematic in FIG. 4.

1. A method for monitoring the health or wellbeing of a user, comprising:
receiving data associated with an output parameter resulting from a physiological response of the user to an input parameter; and
enabling the data to be viewed by the user only after a period of time has elapsed such that the viewable data is not indicative of a real time value of the output parameter.

2. The method of claim 1 wherein the period is pre-defined period.

3. The method of claim 1 or claim 2 in which the input parameter includes one of a time, duration, intensity or quantity of: food or liquid consumption; medicine consumption; exercise or physical movement.

4. The method of any preceding claim in which the output parameter includes one or more of: a blood glucose level; calorific burn rate; calorific intake; heart rate; breathing rate; expiratory and inspiratory volumes; lactic acid levels; drug levels; core temperature; skin temperature; perspiration; and blood pressure.

5. The method of claim 2 in which the input parameter is a quantity of food or liquid consumed by the user and the pre-defined period is a minimum of 1 hour starting from the cessation of food or liquid consumption.

6. The method of claim 1 in which the input parameter is a quantity of food or liquid consumed by the user and the period is determined by in accordance with a sugar level falling back to a normal or rest range, as determined using sensors on or in the user's body.

7. The method of claim 2 in which the input parameter is a duration or intensity of exercise performed by the user and the pre-defined period is a minimum of 5 minutes from cessation of exercise.

8. The method of claim 1 in which the input parameter is a duration or intensity of exercise performed by the user and the period is determined by in accordance with the output parameter falling back to a rest level.

9. The method of any preceding claim further comprising devising an input parameter plan for a subsequent period.

10. The method of claim 9 in which the input parameter plan is determined algorithmically based on historic performance.

11. The method of claim 9 in which the input parameter plan is determine based on user-desired output parameters.

12. The method any preceding claim in which a computer generated value indicating the extent of wellbeing of the user is determined for the user based on the input and/or output parameters.

13. The method of any preceding claim comprising:
encrypting, at a first device, the data regarding the output parameter to provide encrypted data;
sending, from the first device to the second device, the encrypted data and unencrypted data regarding non-confidential user information;
using, at the second device, the unencrypted data to select whether to decrypt the encrypted data.

14. A device for monitoring the health of a user, the device configured to:
receive data associated with an output parameter resulting from a physiological response of the user to an input parameter; and
enable the data to be viewed by the user only after a period of time has elapsed.

15. The device of claim 14 wherein the period is pre-defined.

16. The device of claim 14 or claim 15 in which the input parameter includes one of: a time, duration, intensity or quantity of: food or liquid consumption; medicine consumption; exercise or physical movement.

17. The device of any of claims 14 to 16 in which the output parameter includes one or more of: a blood glucose level; calorific burn rate; calorific intake; heart rate; breathing rate; expiratory and inspiratory volumes; lactic acid levels; drug levels; core temperature; skin temperature; perspiration; blood pressure.

18. The device of any of claims 13 to 16 comprising a sensor for placing on or in a body of the user and for determining the data associated with a physiological response of the user.

19. The device of any of claims 14 to 18 in which the device is a wearable device.

20. The device of any of claims 14 to 19 further configured to devise an input parameter plan for a subsequent period.

21. A method for sharing, between a first device and a second device, data regarding medical or personal information associated with a first user, in which the data contains confidential and non-confidential user information, the method comprising:
encrypting, at the first device, the data regarding the confidential user information to provide encrypted data;
sending, from the first device to the second device, the encrypted data and unencrypted data regarding the non-confidential user information;
using, at the second device, the unencrypted data to select whether to decrypt the encrypted data.

22. The method of claim 20 in which the encrypted data is sent substantially simultaneously with the unencrypted data.

23. The method of any of claim 20 or claim 21 in which confidential information includes one or more of:
user input parameters;
input parameter plans; and
user output parameters of the first user, and
the non-confidential information includes specific user characteristics of the first user.

24. The method of claim 22 in which the specific user characteristics of the first user are compared with specific user characteristics of the second user in order to determine whether or not to decrypt the encrypted data.

25. The method of claim 23 or claim 24 in which the specific user characteristics includes one or more of age, height, weight, race, gender, disease state, body mass index.
